(19) United States
(12) Patent Application Publication
(10) Pub. No.: US 2020/0345671 A1
(43) Pub. Date: Nov. 5, 2020

(54) TOPICAL PHARMACEUTICAL COMPOSITION OF ADAPALENE AND MINOCYCLINE

(71) Applicant: Glenmark Pharmaceuticals Limited, Mumbai (IN)

(72) Inventors: Ulhas R. Dhuppad, Nashik (IN); Madhusudhan Bommagani, Hyderabad, Telangana (IN); Ram Reddy Patlolla, Hyderabad, Telangana (IN); Ganesh Bangar, Ashti, Maharashtra (IN); Atul Chopade, Mumbai, Maharashtra (IN); Gunaji Dhebe, Mumbai, Maharashtra (IN); Muralee Krishna, Navi Mumbai, Maharashtra (IN)

(21) Appl. No.: 16/753,729

(22) PCT Filed: Oct. 24, 2018
(86) PCT No.: PCT/IB2018/058289

§ 371 Date: Apr. 3, 2020

(30) Foreign Application Priority Data
Oct. 24, 2017 (IN) 201721037587
Oct. 26, 2017 (IN) 201721037989

(51) Int. Cl. A61K 31/192; A61K 31/65; A61K 9/00; A61K 9/06; A61K 47/02; A61K 47/34; A61K 47/32; A61K 47/38

(52) U.S. Cl. CPC A61K 31/192; A61K 31/65; A61K 9/0014; A61K 47/38; A61K 47/02; A61K 47/34; A61K 47/32; A61K 9/06

(57) ABSTRACT

A topical pharmaceutical composition comprises adapalene or a pharmaceutical acceptable salt thereof and minocycline or a pharmaceutical acceptable salt thereof. The composition is for topical application and used to treat acne.

### PRIORITY CLAIM

This patent application claims priority to Indian Provisional Patent Application number IN 201721037587 filed on 24 Oct. 2017 and IN 201721037989 filed on 26 Oct. 2017, the contents of which are incorporated by reference herein.

### FIELD OF THE INVENTION

The present invention relates to a pharmaceutical composition (e.g., a fixed-dose combination) comprising adapalene and minocycline. Method of preparing and use of such compositions for the treatment of acne vulgaris are also provided.

### BACKGROUND OF THE INVENTION

Acne vulgaris is an inflammatory disease of the sebaceous glands characterized by an eruption of the skin, often pustular in nature but not supportive. Acne is a common affliction of the adolescent and affects a small but significant percentage of the adult population. Acne involvement results in unsightly lesions, particularly on the face, and in some cases results in severe scarring.

Acne vulgaris, also known as polymorphous juvenile acne, is the commonest form of acne. It comprises four stages:

Stage 1 or comedonal acne, characterized by a large number of open and/or closed comedones and of microcysts.

Stage 2, or papulopustular acne, is of mild to moderate seriousness. It is characterized by the presence of open and/or closed comedones and of microcysts but also of red papules and of pustules. It mainly affects the face and leaves few scars.

Stage 3, or papulocomedonal acne, is more serious and extends to the back, to the thorax and to the shoulders. It is accompanied by a larger number of scars.

Stage 4, or nodulocystic acne, is accompanied by numerous scars. It exhibits nodules and also large painful purplish pustules.

There are various agents for treating acne vulgaris, nevertheless, acne vulgaris is seldom cured and only can be controlled with difficulty. In no case has a treatment designed for any of the aforementioned causes proven to be uniformly effective. Management of acne is challenging, especially considering the chronicity of the disease and the variability in response to treatments.

Adapalene (6-[3-(1-adamantyl)-4-methoxyphenyl]-2-naphthoic acid) is a naphthoic acid derivative with retinoid-like properties. Adapalene possesses anticomedogenic, comedolytic, and anti-inflammatory properties.

Adapalene is marketed under the trademark Differin® at a weight concentration of 0.1%, in the form of an “alcoholic lotion” solution, at a weight concentration of 0.1% and 0.3% in the form of an aqueous gel and at a weight concentration of 0.1% in the form of a cream. These compositions are useful for treating acne.

A fixed-dose combination of adapalene and benzoyl peroxide (BPO) in the form of a gel (Adapalene BPO Gel) has been approved in US under the trade names EPIDUO® and EPIDUO FORTE®. Adapalene BPO Gel is an antibiotic-free combination of adapalene and BPO, a well-established antimicrobial agent.

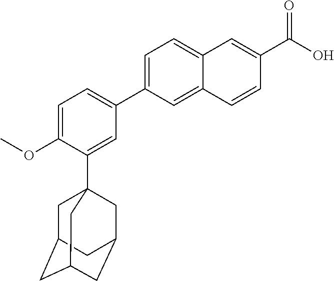 [SMILES: COc1ccc(-c2ccc3cc(C(=O)O)ccc3c2)cc1C12CC3CC(CC(C3)C1)C2]

Minocycline hydrochloride [4S(4α,4aα,5aα,12aα)]-4,7-bis(dimethylamino)-1,4,4a,5,5a,6,11,12a-octahydro-3,10,12,12atetrahydroxy-1,11-dioxo-2-naphthacenecarboxamide mono hydrochloride] is a semi-synthetic derivative of tetracycline. Oral dosage forms of minocycline hydrochloride are available commercially under various trade names. The Approved Drug Products with Therapeutic Equivalence Evaluations (“Orange Book”) lists a number of oral dosage forms of minocycline hydrochloride that are AB-rated to the MINOCIN® brand of minocycline hydrochloride.

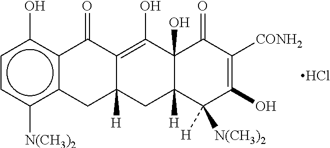

U.S. Publication No. 2014/0147504 discloses that a major challenge in the development of the topical formulation of minocycline has been its chemical nature: it is unstable in solution form and is also sensitive to moisture, temperature, and light. The most commonly reported impurity is formed through the epimerization of minocycline at its C-4 position resulting in the formation of the 4-epi-minocycline stereoisomer of minocycline. No topical minocycline formulation has been approved by the U.S. FDA to date.

U.S. Pat. No. 4,086,332 discloses high potency aqueous doxycycline solutions containing 2-pyrrolidone stabilized by chelation with magnesium compound. The stability of these solutions for therapeutic administration is still further enhanced by the use of antioxidants such as sodium or magnesium formaldehyde sulfoxylate and monothioglycerol at levels of from about 0.01 to 1.0% by weight.

U.S. Pat. No. 4,081,528 discloses aqueous solutions of tetracycline or salts, containing 2-pyrrolidone stabilized by chelation with magnesium compound. The stability of these solutions is further enhanced by the use of antioxidants such as sodium or magnesium formaldehyde sulfoxylate and monothioglycerol at levels of from about 0.01 to 1.0% by weight.

U.S. Pat. No. 4,018,889 discloses oxytetracycline aqueous solutions containing 2-pyrrolidone as a co-solvent stabilized by chelation with pharmaceutically acceptable magnesium compound soluble in said solution. The stability of these solutions for therapeutic administration is still further enhanced by the use of antioxidants such as sodium or magnesium formaldehyde sulfoxylate at levels of from about 0.01 to 1.0% by weight.

GB Patent No. 1,592, 053, discloses a pharmaceutical composition wherein oxytetracycline is stabilized by incorporating it with an alkaline earth metal ion, polyvinyl pyrrolidone and an aliphatic amide and adjusting pH to 5.0 to 7.5. Further, U.S. Pat. No. 3,335,055 discloses a method for stabilization for tetracycline with magnesium ion and a pyridine derivative such as isonicotinamide.

U.S. Publication No. 2016/0279152 discloses a topical composition of minocycline comprising magnesium salt, and a sulfite compound in a non-aqueous solvent.

U.S. Publication No. 2011/0144003 discloses a maintenance therapy regimen for treating acne related diseases. The regimen includes an oral antibiotic drug and a topical treatment with a fixed-dose combination of a retinoid, such as adapalene, and an anti-bacterial agent, such as benzoyl peroxide (BPO).

U.S. Publication No. 2016/0310512 discloses a therapy regimen for the treatment of severe acne related diseases preferentially severe or nodular acne. The therapy regimen adds a topical fixed-dose combination of a retinoid and an anti-bacterial agent, such as benzoyl peroxide, to a course of oral antibiotic therapy using a daily dose of antibiotic ranging from 150 mg to 300 mg per day.

The treatment regimen for acne should be efficacious but also as safe as possible. As a result, there is still an unmet medical need to improve the treatment of acne vulgaris that addresses most of acne causing factors

### SUMMARY OF THE INVENTION

The present invention relates to a pharmaceutical composition (e.g., a fixed-dose combination) comprising adapalene or a pharmaceutically acceptable salt thereof and minocycline or a pharmaceutically acceptable salt thereof. The pharmaceutical composition is for topical application

The topical pharmaceutical composition is useful in the treatment of acne (i.e. mild, moderate and severe). The present invention provides a composition effective in decreasing the inflammatory lesions of acne vulgaris in human patients having such inflammatory lesions.

One embodiment is a topical aqueous gel composition comprising adapalene or a pharmaceutically acceptable salt thereof and minocycline or a pharmaceutically acceptable salt thereof. The composition may comprise from about 1 to about 99% by weight of one or more aqueous solvents. In one embodiment, the aqueous solvents may be present in amount ranging from about 10 to about 95% by weight, based on the total weight of the topical composition.

In another embodiment composition is a topical non-aqueous gel composition comprising adapalene or a pharmaceutically acceptable salt thereof and minocycline or a pharmaceutically acceptable salt thereof. The composition may comprise from about 1 to about 99% by weight of one or more non-aqueous solvents, based on total weight of the topical composition.

In yet another embodiment, the present invention provides a method of treating acne topically with a pharmaceutical composition comprising a combination of: (a) adapalene or a pharmaceutically acceptable salt thereof; and (b) minocycline or a pharmaceutically acceptable salt thereof. In one embodiment, the clinical efficacy of the combination is greater than that of either the adapalene component or the minocycline component administered alone.

In yet another embodiment, the present invention provides a method of treating acne comprising topically administering a pharmaceutical composition comprising a combination of (a) adapalene or a pharmaceutically acceptable salt thereof and (b) minocycline or a pharmaceutically acceptable salt thereof.

The topical composition may be provided in the form of a semi-solid or liquid composition, including, but not limited to, a topical cream, gel, lotion, ointment, liniment, paste, oil-in-water emulsion, water-in-oil emulsion, and foam. Preferably the topical pharmaceutical composition is in the form of a gel. The semi-solid or liquid composition can be an aqueous or a non-aqueous composition. The gel can be an aqueous or a non-aqueous composition.

In one embodiment, the topical composition comprises from about 0.01% to about 0.3% w/w adapalene (e.g., 0.1% or 0.3% adapalene) or a pharmaceutically acceptable salt thereof and from about 0.01 to about 20% w/w minocycline or a pharmaceutically acceptable salt thereof (e.g. 1%, 2% or 4% minocycline hydrochloride), a magnesium compound and one or more solvents.

In one embodiment, the topical composition comprises from about 0.01% to about 0.3% w/w of adapalene or a pharmaceutically acceptable salt thereof and from about 0.01 to about 20% w/w minocycline or a pharmaceutically acceptable salt thereof and one or more solvents, based on total weight of the topical composition.

In another embodiment, the topical composition comprises from about 0.1% to about 0.3% w/w adapalene and from about 0.1 to about 10% w/w minocycline, a magnesium compound and one or more aqueous solvents.

In another embodiment, the topical composition comprises from about 0.1% to about 0.3% w/w adapalene or a pharmaceutically acceptable salt thereof and from about 0.1 to about 10% w/w minocycline or a pharmaceutically acceptable salt thereof and one or more non-aqueous solvent, based on total weight of the topical composition.

In yet another embodiment, the present invention provides a method of treating acne by topically administering a pharmaceutical composition of the present invention.

In another aspect, the present invention provides a method of treating acne topically with a pharmaceutical composition comprising a combination of (a) adapalene or a pharmaceutically acceptable salt thereof and (b) minocycline or a pharmaceutically acceptable salt thereof, according to any of the embodiments described herein.

### DESCRIPTION OF THE INVENTION

### Definitions

The term “acne lesions” refers to non-inflammatory lesions (open and closed comedones) and inflammatory lesions (papules, pustules, nodules and cysts) caused by acne. Preferably, the inflammatory lesions are treated with the topical composition of the invention.

The term “patient” includes adolescents, teenagers, and adults (for instance, 18 years and older).

6-[3-(1-adamantyl)-4-methoxyphenyl]-2-naphthoic acid (referred to herein as adapalene) is a naphthoic acid derivative with retinoid and anti-inflammatory properties. Adapalene used in the compositions of the present invention may be in the form of its free acid or a pharmaceutically acceptable salt thereof.

Minocycline is [4S(4α,4aα,5aα,12aα)]-4,7-bis(dimethylamino)-1,4,4a,5,5a,6,11,12a-octahydro-3,10,12,12atetrahydroxy-1,11-dioxo-2-naphthacenecarboxamide. Minocycline used in the compositions of the present invention may be in the form of the free base or its acid addition salt with a pharmaceutically acceptable acid, such as hydrochloric acid, sulfuric acid, trichloroacetic acid and the like.

The term “fixed dose combination” refers to a combination wherein the active principles are combined at fixed doses in the same vehicle or medium (single formula) that delivers them together to the point of application. Preferably, the pharmaceutical composition in the form of a fixed combination is a gel; in this case, the two active principles are dispersed and intimately mixed, during the manufacture, in the same vehicle, which delivers them together during the application of the gel. In one or more embodiment the two active principles may be mixed upon dispensing.

The term “treating” or “treatment” refers to obtaining a desired pharmacological and/or physiological effect. The effect can be prophylactic or therapeutic, which includes achieving, partially or substantially, one or more of the following results: partially or totally reducing the extent of the disease, disorder or syndrome; ameliorating or improving a clinical symptom or indicator associated with the disorder; delaying, inhibiting or decreasing the likelihood of the progression of the disease, disorder or syndrome; or partially or totally delaying, inhibiting or reducing the likelihood of the onset or development of disease, disorder or syndrome.

The term “topical” and its derivatives as used herein refer to directly laying on or spreading on the skin in need of the treatment, e.g., by use of the hands or an applicator.

The term “efficacy” as used herein refers to the extent to which a treatment produces a beneficial result, e.g., an improvement in one or more symptoms of the disease. In one embodiment, efficacy is evaluated by lesion counting, which is a current practice to assess severity of acne.

The term “stable” refers to a minimal change in physical and chemical properties of the composition over time relative to when it is manufactured. Stability over time may be evaluated based on pre-defined criteria including assay of active and related compounds, appearance, color, and pH.

The term “aqueous” as used herein, refers to a liquid that has a significant portion of water or water miscible solvent, and in one embodiment it is at least about 90% based on total weight of the topical composition. In one embodiment, it is a liquid containing at least about 60% by weight water. In another embodiment aqueous solvent may include at least about 50%, or, at least about 40% by weight water.

The term “non-aqueous” as used herein, means that the composition contains no, or essentially no free or unassociated or absorbed water. In certain other preferred embodiments the composition is “substantially non-aqueous” or “substantially waterless”. The term “substantially non-aqueous” or “substantially waterless” is intended to indicate that the composition has a water content below about 50%, preferably below about 25%, such as below about 10%, below about 5% or below about 1%.

In one or more embodiment aqueous solvent may include a mixture of water and water miscible solvent. Suitable water miscible solvents include, but are not limited to, methanol, ethanol, n-propanol, isopropanol, monomethyl ether of ethylene glycol, monoethyl ether of ethylene glycol, THF, dioxane, acetone, acetonitrile, dimethyl ether of ethylene glycol and mixtures of any two or more of the foregoing.

Suitable magnesium compounds include those that are pharmaceutically acceptable material, for example, magnesium chloride, magnesium acetate, magnesium sulfate, magnesium carbonate, and magnesium gluconate, magnesium chloride hexahydrate. One preferable magnesium compound is magnesium chloride. The magnesium compound is preferably in an amount sufficient to improve the stability of the minocycline. In one embodiment, the magnesium compound is present in an amount ranging from about 0.1 to about 15% by weight, preferably, from about 0.1 to about 10% by weight based on the total weight of the composition.

One advantage of the compositions described herein is the stability of such compositions, particularly with respect to discoloration over time and the amount of impurities, including breakdown products of the active material, such as 4-epi-minocycline.

One embodiment is a stable topical gel formulation containing adapalene or a pharmaceutically acceptable salt thereof and minocycline or a pharmaceutically acceptable salt thereof where the formation of one or more impurities from the adapalene or minocycline are controlled or minimized to very low levels for example below 3%, 1%, 0.5%, or 0.2% by weight for each impurity. In one embodiment, the impurity 4-epi minocycline remains below 3% w/w (i.e. by weight of minocycline hydrochloride) when stored at 40° C. and 75% relative humidity (RH) for one month, e.g., as determined by High Performance Liquid Chromatography (HPLC) analysis (column: Hypersil BDS C18 (4.6 mm×250 mm, 5μ) developing agent: water: acetonitrile: tetrahydrofuran, room temperature), and ultraviolet absorbance (275 nm).

In one embodiment, the invention provides a stable topical gel composition wherein all the impurities are controlled, especially the impurity 4-epi minocycline, to a very low level, for example below 3%, 1%, 0.5%, or 0.2% by weight for each impurity.

In another embodiment, the content of 4-epi minocycline is below about 1.2% w/w, when stored at 40° C. and 75% RH for one month.

In one embodiment, the topical composition comprises from about 0.01 or 0.1 to about 15% w/w adapalene or a pharmaceutically acceptable salt thereof and from about 0.1 to 20% w/w minocycline or a pharmaceutically acceptable salt thereof, a magnesium compound and a solvent, based on the total weight of the topical composition.

In one embodiment, the topical composition comprises from about 0.01 or 0.1 to about 15% w/w adapalene or a pharmaceutically acceptable salt thereof from about 0.1 to about 15% w/w minocycline or a pharmaceutically acceptable salt thereof and a solvent based on the total weight of the topical composition.

In another embodiment, the topical composition comprises from about 0.01 or 0.1 to about 15% w/w adapalene or a pharmaceutically acceptable salt thereof and from about 0.1 to 15% w/w minocycline or a pharmaceutically acceptable salt thereof a magnesium compound, and a solvent based on the total weight of the topical composition.

In another embodiment, the topical composition comprises from about 0.01 or 0.1 to about 15% w/w adapalene or a pharmaceutically acceptable salt thereof, from about 0.1 to about 10% w/w minocycline or a pharmaceutically acceptable salt thereof and a solvent based on the total weight of the topical composition.

In yet another embodiment, the topical composition comprises from about 0.1 to about 0.3% w/w adapalene or a pharmaceutically acceptable salt thereof and from about 0.1 to 10% w/w minocycline or a pharmaceutically acceptable salt thereof a magnesium compound, and a solvent based on the total weight of the topical composition.

In yet another embodiment, the topical composition comprises from about 0.1 to about 0.3% w/w adapalene or a pharmaceutically acceptable salt thereof, from about 0.1 to about 10% w/w minocycline or a pharmaceutically acceptable salt thereof and a solvent based on the total weight of the topical composition.

In yet another embodiment, the topical composition comprises from about 0.01 to about 1.0% w/w adapalene and from about 0.01 to about 10% w/w minocycline, a magnesium compound and a solvent based on the total pharmaceutical composition.

In yet another embodiment, the topical composition comprises from about 0.01 to about 1.0% w/w adapalene or a pharmaceutically acceptable salt thereof and from about 0.01 to about 20% w/w minocycline or a pharmaceutically acceptable salt thereof, based on the total weight of the topical composition.

One embodiment is a topical aqueous gel composition comprising adapalene or a pharmaceutically acceptable salt thereof and minocycline or a pharmaceutically acceptable salt thereof. The composition may comprise from about 1 to about 99% by weight of one or more aqueous solvents, based upon 100% total weight of the topical composition, wherein the aqueous solvent is water.

In one embodiment, the topical composition comprises from about 0.1 to about 0.3% w/w adapalene or a pharmaceutically acceptable salt thereof, from about 0.1 to about 10.0% w/w minocycline or a pharmaceutically acceptable salt thereof, and a solvent, based on the total weight of topical composition. The solvent may be aqueous, non-aqueous, or mixture thereof. In one embodiment, the solvent is non-aqueous.

In another embodiment the topical composition comprises a fixed dose combination of about 0.1% w/w adapalene or a pharmaceutically acceptable salt thereof and about 2% w/w minocycline or a pharmaceutically acceptable salt thereof, a magnesium compound and one or more solvents, based on total weight of the topical composition.

In another embodiment the topical composition comprises a fixed dose combination of about 0.1% w/w adapalene or a pharmaceutically acceptable salt thereof and about 2% w/w minocycline or a pharmaceutically acceptable salt thereof and one or more solvents, based on total weight of the topical composition.

In yet another embodiment the topical composition comprises a fixed dose combination of about 0.1% w/w adapalene or a pharmaceutically acceptable salt thereof and about 4% w/w minocycline or a pharmaceutically acceptable salt thereof a magnesium compound and one or more solvents, based on total weight of the topical composition.

In another embodiment the topical composition comprises a fixed dose combination of about 0.1% w/w adapalene or a pharmaceutically acceptable salt thereof and about 42% w/w minocycline or a pharmaceutically acceptable salt thereof and one or more solvents, based on total weight of the topical composition.

In one embodiment, the topical composition comprises from about 0.1 to about 0.3% w/w adapalene or a pharmaceutically acceptable salt thereof, from about 0.1 to about 10.0% w/w minocycline or a pharmaceutically acceptable salt thereof, a magnesium compound, a gelling agent and solvent, based on total weight of total composition. The solvent may be an aqueous solvent.

In another embodiment, the topical composition comprises a fixed dose combination comprises from about 0.1% to about 1% w/w adapalene or a pharmaceutically acceptable salt thereof, from about 0.01 to about 20% w/w minocycline or a pharmaceutically acceptable salt thereof, about 10 to about 99% by weight of non-aqueous solvent or mixtures, wherein the content of 4-epi minocycline is below 3%, when stored at 40° C. and 75% relative humidity for one month as determined by HPLC analysis.

One embodiment is a topical non-aqueous gel composition comprising adapalene or a pharmaceutically acceptable salt thereof and minocycline or a pharmaceutically acceptable salt thereof. The composition may comprise from about 1 to about 99% by weight of one or more non-aqueous solvents, based on total weight of the topical composition. In one or more embodiment the composition may comprise from about 1 to about 85%, preferably from about 1 to about 75%, by weight of one or more non-aqueous solvents, based on total weight of the topical composition.

In one embodiment, the topical composition is a non-aqueous gel comprising from about 0.1% to about 0.3% w/w adapalene or a pharmaceutically acceptable salt thereof, from about 1.0 to about 10% w/w minocycline or a pharmaceutically acceptable salt thereof, a gelling agent, and a non-aqueous solvent.

Suitable gelling agents include, but are not limited to, a cellulose ether, such as methylcellulose, hydroxypropyl cellulose, hydroxypropyl methylcellulose, hydroxyethyl cellulose, hydroxypropyl methyl cellulose, methylhydroxyethylcellulose, methylhydroxypropylcellulose, hydroxyethylcarboxymethylcellulose, carboxymethyl cellulose, carboxymethylcellulose, carboxymethylhydroxyethylcellulose, and cationic celluloses, carbomer (homopolymer of acrylic acid is crosslinked with an allyl ether pentaerythritol, an allyl ether of sucrose, or an allyl ether of propylene), such as Carbopol® 934, Carbopol® 940, Carbopol® 941, Carbopol® 980 and Carbopol® 981, acrylamide / sodium acryloyldimethyltaurate copolymer/isohexadecane/polysorbate 80 (available as Sepineo P 600 from SEPPIC Inc. of Fairfield, N.J.), Pemulen, Klucel, and aluminum starch octenylsuccinate (ASOS) or other derivatized polymers, polyethylene glycol, having molecular weight of 1000 or more (e.g., PEG 1,000, PEG 4,000, PEG 6,000 and PEG 10,000), locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, amine-bearing polymers such as chitosan; acidic polymers obtainable from natural sources, such as alginic acid and hyaluronic acid or their salts such as sodium alginate and sodium hyaluronate; chemically modified starches, carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride, cyclopentasiloxane and dimethicone cross polymer (ST-Elastomer 9041 and ST-Elastomer 10). One or more gelling agents in any combination can be used. The gelling agent may be present in amounts ranging from about 0.05 to about 50% w/w, more specifically from about 0.05 to about 25% w/w. For example, the gelling agent may be present in an amount ranging from about 0.05% w/w to about 10% w/w.

In one embodiment, the gelling agent(s) is present in the composition in amount ranging from about 0.05 to about 80% w/w (based on total weight of the topical composition). The gelling agent(s) may be present in an amount ranging from about 0.05 to about 75% w/w, more specifically from about 0.05 to about 65% w/w (based on total weight of the topical composition)

In one embodiment, the gelling agent comprises one or more carbomer and/or cellulose derivatives.

In yet another embodiment, the topical composition comprises from about 0.1 to about 0.3% w/w adapalene or a pharmaceutically acceptable salt thereof and from about 0.1 to 10.0% w/w minocycline or a pharmaceutically acceptable salt thereof, a magnesium compound, a gelling agent, and a solvent.

The topical composition may contain one or more additional ingredients such as a penetration enhancer, chelating agent, a buffer agent, an anti-oxidant, and a preservative to provide a physicochemically stable composition.

In one embodiment, the topical gel composition comprises from about 0.1% to about 0.3% w/w adapalene, from about 1.0 to about 10% w/w minocycline, a magnesium compound, a gelling agent, water, and one or more additional excipients, such as a penetration enhancer, chelating agent, buffering agent, antioxidant, preservative, or any combination of any of the foregoing.

In one embodiment, the topical gel composition comprises from about 0.1% to about 0.3% w/w adapalene or a pharmaceutically acceptable salt thereof, from about 1.0 to about 10% w/w minocycline or a pharmaceutically acceptable salt thereof, a surfactant, a gelling agent, a penetration enhancer, a solvent and optionally other additional excipients such as a chelating agent, a preservative, and a buffering agent.

In one embodiment the topical composition comprises a fixed dose combination comprises from about 0.1% to about 1% w/w adapalene or a pharmaceutically acceptable salt thereof, from about 0.01 to about 20% w/w minocycline or a pharmaceutically acceptable salt thereof, about 10 to about 99% by weight of non-aqueous solvent or mixtures, and about 0.05 to about 80% by weight of a gelling agent.

In another embodiment, the topical composition comprises a fixed dose combination comprises from about 0.1% to about 1% w/w adapalene or a pharmaceutically acceptable salt thereof, from about 0.01 to about 20% w/w minocycline or a pharmaceutically acceptable salt thereof, about 10 to about 99% by weight of non-aqueous solvent or mixtures, about 0.05 to about 80% by weight of a gelling agent, wherein the content of 4-epi minocycline is below 3%, when stored at 40° C. and 75% relative humidity for one month as determined by HPLC analysis.

In another embodiment, the topical gel composition has a viscosity of not more than about 15,000 cP, preferably between about 100 and about 12,000 cP, and more preferably between about 300 and about 10,000 cP. The viscosity may be determined at room temperature (20-25° C) using a Brookfield Viscometer Model CAP 2000 or Cone Plate Wells Brookfield Viscometer.

The topical composition may include one or more co-solvents such as, without limitation, volatile organic solvents (e.g. alcohols such as ethanol, propanol or butanol), benzoyl alcohol, non-volatile organic solvents (e.g. amides such as pyrrolidones; polyol ethers such as glycol ethers; polyols such as glycols; and derivatives thereof) and water or mixtures thereof.

Suitable penetration enhancers include, but are not limited to, dimethyl isosorbide, glycerol (glycerin), propylene glycol, hexylene glycol, diethylene glycol, propylene glycol n-alkanols, terpenes, di-terpenes, tri-terpenes, limonene, terpene-ol, 1-menthol, dioxolane, ethylene glycol, other glycols, oleyl alcohol, alpha-hydroxy acids, such as lactic acid and glycolic acid, sulfoxides, such as dimethylsulfoxide (DMSO), dimethylformanide, methyl dodecyl sulfoxide, dimethylacetamide, azone (1-dodecylazacycloheptan-2-one), 2-(n-nonyl)-1,3-dioxolane, alkanols, such as dialkylamino acetates, dimethylformanide, methyl dodecyl sulfoxide, dimethylacetamide, dimethylisosorbide, monooleate of ethoxylated glycerides (with 8 to 10 ethylene oxide units), azone (1-dodecylazacycloheptan-2-one), 2-(n-nonyl)-1,3-dioxolane, esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid, and any combination of any of the foregoing.

The penetration enhancer may be present in an amount ranging from about 0.05 to 30 w/w %, specifically from about 0. 1% w/w to about 25% w/w, and more specifically from about 5% to about 20% w/w.

In one embodiment, the penetration enhancer comprises dimethyl isosorbide and/or polyethylene glycol (PEG) or a PEG derivative, for example, in an amount ranging from about 0.05 to 30 w/w % (based on the total weight of the topical composition).

The penetration enhancer may be present in an amount ranging from about 0.5 to about 10% w/w, such as from about 0.5 to about 5% w/w, or from about 0.5 to about 3% w/w (based on the total weight of the topical composition).

Suitable chelating agents include, but are not limited to, ethylenediamine tetracetic acid (EDTA), commercially available both as the free acid and as various salts, for example, disodium EDTA, tetrasodium EDTA, dipotassium EDTA, and calcium disodium EDTA. Other suitable chelators include the naturally occurring amino acid L-cysteine, HSCH₂ CH(NH₂)CO₂ H, and its acetylated derivative N-acetyl-L-cysteine, HSCH₂-CH(NHCOCH₃)CO₂ H, commonly referred to as NAC.

Suitable buffering agents include, but are not limited to, citrate/citric acid, acetate/acetic acid, phosphate/phosphoric acid, formate/formic acid, propionate/propionic acid, lactate/lactic acid, carbonate/carbonic acid, ammonium/ammonia, edentate/edetic acid, and derivatives thereof, sodium hydroxide, sodium chloride, potassium chloride, potassium carbonate and mixtures thereof. Suitable antioxidants include, for example, butylated hydroxyanisole (BHA), butylated hydroxytoluene (BHT), ascorbic acid, tocopherols and tocotrienols (e.g. vitamin E), carotenes, flavonoids (e.g. quercetin) or mixtures, tertiary butyl hydroquinone, propyl gallate, α-tocopherol, sodium metabisulfite, glutathione, N-acetylcysteine, thioproline, taurine, sodium selenite, sulfurous acid salts and organic esters such as bisulfites, pyrosulfites, metabisulfites, and sulfites, and any combination of any of the foregoing. The antioxidant may be present in amounts ranging from about 0.005% to about 3.0% by weight of the composition.

Suitable preservatives include, but are not limited to, benzyl alcohol, diazolidinyl urea, methyl paraben, ethyl paraben, propyl paraben, butyl paraben, phenoxyethanol, sorbic acid, and salts thereof such as potassium sorbate, benzoic acid and salts thereof such as sodium benzoate, and any combination of any of the foregoing.

Suitable surfactants include, but are not limited to, polysorbates, polyoxyethylene (20) sorbitan monostearate, polyoxyethylene (20) sorbitan monooleate, a polyoxyethylene fatty acid ester (e.g. polyoxyethylene (8) stearate (Myrj 45), polyoxyethylene (20) stearate (Myrj 49), polyoxyethylene (40) stearate (Myrj 52), polyoxyethylene (100) stearate (Myrj 59)), a polyoxyethylene alkynyl ether, polyoxyethylene cetyl ether, polyoxyethylene palmityl ether, polyethylene oxide hexadecyl ether, polyethylene glycol cetyl ether (e.g., Brij 38, Brij 52, Brij 56 and Brij W1), a sucrose ester, a partial ester of sorbitol, sorbitan monolaurate, sorbitan monolaurate a monoglyceride, a diglyceride, isoceteth-20, a sucrose ester, steareth 2, glyceryl monostearate/PEG 100 stearate, glyceryl stearate, steareth-21, peg 40 stearate, polysorbate 60, polysorbate 80, sorbitan stearate, laureth 4, sorbitan monooleate, ceteareth 20, steareth 20, ceteth 20, macrogol cetostearyl ether, ceteth 2, peg-30 dipolyhydroxystearate, sucrose distearate, polyoxyethylene (100) stearate, PEG 100 stearate, PEG 40 stearate, laureth 4, cetomacrogol ether, cetearyl alcohol, cetearyl glucoside, oleyl alcohol, steareth-2, diisopropyl adipate, capric/caprilic triglicerides, polysorbate 20; Montanov 68 (cetearyl alcohol (and) cetearyl glucoside), Poloxamer 182, Poloxamer 124 , Simusol 165 (glyceryl stearate and PEG-100 stearate), methyl glucose sequistearate, PEG 30 dipolyhydroxystearate, sucrose stearic acid esters, sorbitan laureth, sorbitan stearate, and any combination of the any of the foregoing.

In one preferred embodiment, the surfactant comprises a polysorbate and/or PEG 30 dipolyhydroxystearate, for example, in an amount ranging from about 0.05 to 15% w/w (based on the total weight of the topical composition).

In one embodiment, there is provided an aqueous gel composition for topical application which comprises adapalene or a pharmaceutically acceptable salt thereof in an amount ranging from about 0.01 to about 1.0% w/w and minocycline or a pharmaceutically acceptable salt thereof in an amount ranging from about 0.01 to 20.0%, a magnesium compound, a gelling agent, and water, based on total weight of the composition.

In another embodiment, a stable topical composition comprises a fixed dose combination comprising about 0.1% to about 0.3% w/w adapalene or a pharmaceutically acceptable salt thereof, about 0.1 to about 10% w/w minocycline or a pharmaceutically acceptable salt thereof, about 10 to about 99% by weight of non-aqueous solvent or mixtures, about 0.05 to about 80% by weight of a gelling agent, about 0.05 to about 30% by weight of a penetration enhancer, wherein the composition is waterless.

In another embodiment, the topical composition is an aqueous gel comprising from about 0.1% to about 0.3% w/w adapalene or a pharmaceutically acceptable salt thereof and from about 0.1 to about 10% w/w minocycline or a pharmaceutically acceptable salt thereof, about 0.01 to about 0.5% w/w of magnesium compound, about 0.05% w/w to about 10% w/w of a gelling agent, and about 10% to about 95% of water.

In one embodiment, the topical composition is a non-aqueous gel comprising from about 0.1% to about 0.3% w/w adapalene or a pharmaceutically acceptable salt thereof from about 1.0 to about 10% w/w minocycline or a pharmaceutically acceptable salt thereof, a gelling agent, and a non-aqueous solvent, where the composition is free or substantially free of a penetration enhancer.

In yet another embodiment, the aqueous gel composition comprises from about 0.1% to about 0.3% w/w adapalene or a pharmaceutically acceptable salt thereof and from about 0.1 to about 10% w/w minocycline or a pharmaceutically acceptable salt thereof, about 0.01 to about 0.5% w/w of magnesium compound, about 0.05% w/w to about 10% w/w of a gelling agent, and about 10% to about 95% of water.

In yet another embodiment, the topical composition comprises from about 0.1 to about 0.3% w/w adapalene or a pharmaceutically acceptable salt thereof and from about 0.1 to 10.0% w/w minocycline or a pharmaceutically acceptable salt thereof, a gelling agent, a penetration enhancer, and a solvent.

In yet another embodiment, the stable aqueous gel composition comprises about 0.1% to about 0.3% w/w adapalene or a pharmaceutically acceptable salt thereof and about 1.0 to about 10% w/w minocycline or a pharmaceutically acceptable salt thereof, about 0.1% to about 3% by weight of magnesium chloride, about 0.1% to about 0.2% by weight of sodium sulfite, about 4% to about 10% by weight of propylene glycol, about 4% to about 10% by weight of glycerin, about 2% to about 4% by weight of acrylamide/sodium acryloyldimethyltaurate copolymer/isohexadecane/polysorbate 80, and about 70% to about 85% of purified water.

In one embodiment, the topical composition comprises from about 0.1 to about 0.3% w/w adapalene or a pharmaceutically acceptable salt thereof, from about 0.1 to about 10% w/w minocycline or a pharmaceutically acceptable salt thereof, a gelling agent, a penetration enhancer, a surfactant, and from about 1 to about 99% of a solvent. The composition may include both active ingredients stabilized in a gelling matrix or in a non-aqueous base.

In another embodiment, the topical gel composition has a viscosity of not more than about 15,000 cP, preferably between about 100 and about 12,000 cP, and more preferably between about 300 and about 10,000 cP. The viscosity may be determined at room temperature (20-25° C.) using a Brookfield Viscometer Model CAP 2000 or Cone Plate Wells Brookfield

Viscometer.

In yet another embodiment, the stable topical gel composition comprises a fixed dose combination comprising about 0.1% to about 0.3% w/w adapalene or a pharmaceutically acceptable salt thereof, about 0.1 to about 10% w/w minocycline or a pharmaceutically acceptable salt thereof, about 10 to about 99% by weight of non-aqueous solvent or mixtures, about 0.05 to about 80% by weight of a gelling agent, wherein the topical gel has a viscosity between about 100 to about 12,000 cP.

In one embodiment, the stable aqueous gel composition comprises about 0.1% to about 0.3% w/w adapalene or a pharmaceutically acceptable salt thereof and about 1.0 to about 10% w/w minocycline or a pharmaceutically acceptable salt thereof, about 0.1% to about 3% by weight of magnesium chloride, about 0.1% to about 0.2% by weight of sodium sulfite, about 4% to about 10% by weight of propylene glycol, about 4% to about 10% by weight of glycerin, about 2% to about 4% by weight of acrylamide/sodium acryloyldimethyltaurate copolymer/isohexadecane/polysorbate 80, and about 70% to about 85% of purified water, wherein the content of 4-epi minocycline is below 3%, when stored at 40° C. and 75% relative humidity for one month as determined by HPLC analysis.

In one embodiment, the stable topical gel composition comprises fixed dose combination comprising about 0.1% to about 0.3% w/w adapalene or a pharmaceutically acceptable salt thereof, about 0.1 to about 10% w/w minocycline or a pharmaceutically acceptable salt thereof, about 10 to about 99% by weight of non-aqueous solvent or mixtures, about 0.05 to about 80% by weight of a gelling agent, about 0.05 to about 30% by weight of a penetration enhancer, wherein the content of 4-epi minocycline is below 3%, when stored at 40° C. and 75% relative humidity for one month as determined by HPLC analysis.

In one embodiment, the aqueous gel composition comprises about 0.1% to about 0.3% w/w adapalene or a pharmaceutically acceptable salt thereof and about 1.0 to about 10% w/w minocycline or a pharmaceutically acceptable salt thereof, about 0.1% to about 3% by weight of magnesium chloride, about 0.1% to about 0.2% by weight of sodium sulfite, about 4% to about 10% by weight of propylene glycol, about 4% to about 10% by weight of glycerin, about 1% to about 2.5% by weight of sodium hyaluronate, and about 70% to about 85% of purified water.

In another embodiment, the compositions described herein are waterless, i.e., the composition contains no or substantially no (e.g., less than 2% by weight), free or unassociated or absorbed water.

The non-aqueous gel compositions may include, for example, at least one non-aqueous solvent, for example, selected from a fatty alcohol, a fatty acid, and/or naturally occurring waxes.

Suitable non-aqueous solvents include, but are not limited to, an organic carrier selected from mineral oil, light mineral oil, triglycerides, medium chain triglyceride (MCT) oil, capric/caprylic triglyceride, alkyl esters of fatty acids such as isopropyl palmitate, isopropyl myristate, isopropyl isostearate, poly propylene glycol 15-stearly ether, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, maleated soybean oil, unsaturated or polyunsaturated oils, such as olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils; essential oils; and silicone oils, such as dimethicone, cyclomethicone, polyalkyl siloxane, polyaryl siloxane, polyalkylaryl siloxane, a polyether siloxane copolymer and a poly(dimethylsiloxane)-(diphenyl-siloxane) copolymer and a polypropylene glycol alkyl ether (PPG 15 stearyl ether), dimethyl isosorbide, glycerol, propylene glycol, hexylene glycol, diethylene glycol, propylene glycol n-alkanols, terpenes, di-terpenes, tri-terpenes, limonene, terpene-ol, 1-menthol, dioxolane, ethylene glycol, other glycols, oleyl alcohol, alpha-hydroxy acids, such as lactic acid and glycolic acid, sulfoxides, such as dimethylsulfoxide (DMSO), dimethylformanide, methyl dodecyl sulfoxide, dimethylacetamide, azone (1-dodecylazacycloheptan-2-one), 2-(n-nonyl)-1,3-dioxolane, alkanols, such as dialkylamino acetates, capric/caprylic triglyceride, and any combination of any of the foregoing.

Suitable liquid and waxes include, but are not limited to, isostearic acid, oleic acid, oleyl alcohol, stearic acid, cetyl alcohol, stearyl alcohol, erucic acid, linoleic acid, arachidonic acid, lauric acid, myristic acid, palmitic acid, stearic acid, behenic acid, oleic acid, 12-hydroxystearic acid, undecylenic acid, tall acid, isostearic acid, linoleic acid, linolenic acid, eicosapentaenoic acid (EPA), docosahexaenoic acid (DHA), animal waxes, spermaceti, lanolin (wool wax), insect waxes, beeswax, silky wax, chinese wax, vegetable waxes, candelilla wax, castor wax, jojoba oil, rice bran wax; petroleum waxes, paraffin waxes, microcrystalline wax; synthetic waxes, polyethylene waxes, Fischer-Tropsch waxes, chemically modified and substituted waxes; and mineral waxes, and any combination of any of the foregoing.

In one preferred embodiment, a solvent used in the present invention is a silicone oil such as cyclomethicone (octamethylcyclotetrasiloxane), isopropyl palmitate, isopropyl myristate and ethylene glycol or any mixture thereof.

The amount of non-aqueous solvent may range from about 1 to about 99% by weight, based on the total weight of the topical composition. In one embodiment, the amount of non-aqueous solvent ranges from about 1% to about 65%, more preferably from about 1% to about 60%, based on the total weight of the topical composition.

The topical composition may include one or more co-solvents such as, without limitation, volatile organic solvents (e.g. alcohols such as ethanol, propanol or butanol), benzoyl alcohol, nonvolatile organic solvents (e.g. amides such as pyrrolidones; polyol ethers such as glycol ethers; polyols such as glycols; and derivatives thereof) or any combination of any of the foregoing.

The topical composition may contain additional ingredients to provide aesthetic, moisturizing (emollients) and/or anti-inflammatory benefits to the skin, such as moisturizers and emollients. A moisturizer or emollient may provide a layer of oil on the surface of the skin to slow water loss and thus increase the moisture content of the stratum corneum and/or act as a humectant which, upon introduction into the stratum corneum, increases its water holding capacity. Suitable emollients include, but are not limited to, octyldodecanol, cetyl alcohol, stearyl alcohol and cetearyl alcohol, hydrocarbons, e.g., petrolatum and light mineral oil, acetylated lanolin and mixtures thereof.

The topical composition may contain additional ingredients such as a chelating agent, a buffer agent, an anti-oxidant, a preservative, and any combination of any of the foregoing to provide a physicochemically stable composition.

In another embodiment, the aqueous gel composition comprises about 0.1% to about 0.3% w/w adapalene or a pharmaceutically acceptable salt thereof and about 1.0 to about 10% w/w minocycline or a pharmaceutically acceptable salt thereof, about 0.1% to about 3% by weight of magnesium chloride, about 0.1% to about 0.2% by weight of sodium sulfite, about 4% to about 10% by weight of propylene glycol, about 4% to about 10% by weight of glycerin, about 1% to about 2.5% by weight of sodium hyaluronate, and about 70% to about 85% of purified water.

In another embodiment, there is provided a non-aqueous gel composition for topical application which comprises adapalene in an amount ranging from about 0.01 to about 1.0% adapalene or a pharmaceutically acceptable salt thereof and minocycline in an amount ranging from about 0.01 to 20.0% or a pharmaceutically acceptable salt thereof, a gelling agent, and a non-aqueous solvent.

In yet another embodiment, the aqueous gel composition comprises about 0.1% to about 0.3% w/w adapalene or a pharmaceutically acceptable salt thereof and about 1.0 to about 10% w/w minocycline or a pharmaceutically acceptable salt thereof, about 0.1% to about 3% by weight of magnesium chloride, about 0.1% to about 0.2% by weight of sodium sulfite, about 4% to about 10% by weight of propylene glycol, about 4% to about 10% by weight of glycerin, about 0.5% to about 1.5% by weight of hydroxyl propyl methyl cellulose, and about 70% to about 85% of purified water.

In one embodiment, the topical composition is a non-aqueous gel comprising from about 0.1% to about 0.3% w/w adapalene or a pharmaceutically acceptable salt thereof, from about 0.1 to about 10% w/w minocycline or a pharmaceutically acceptable salt thereof, about 40 to about 70% of gelling agent, and about 10% to about 40% of non-aqueous solvent comprising light mineral oil, cetostearyl alcohol and cyclomethicone. In one or more embodiments, the topical compositions are free of a wax.

In one embodiment, the aqueous gel composition comprises about 0.1% to about 0.3% w/w adapalene or a pharmaceutically acceptable salt thereof and about 1.0 to about 10% w/w minocycline or a pharmaceutically acceptable salt thereof, about 0.1% to about 3% by weight of magnesium chloride, about 0.1% to about 0.2% by weight of sodium sulfite, about 4% to about 10% by weight of propylene glycol, about 4% to about 10% by weight of glycerin, about 1% to about 1.75% by weight of hydroxyl ethyl cellulose, and about 70% to about 85% of purified water.

In another embodiment, the non-aqueous gel comprises from about 0.1% to about 0.3% w/w adapalene or a pharmaceutically acceptable salt thereof, from about 0.1 to about 10% w/w minocycline or a pharmaceutically acceptable salt thereof, about 40 to about 70% of gelling agent, and from about 10% to about 40% of a non-aqueous solvent comprising light mineral oil, cetostearyl alcohol, a wax such as silky wax or bees wax, and cyclomethicone.

In another embodiment, the aqueous gel composition comprises about 0.1% w/w adapalene or a pharmaceutically acceptable salt thereof and about 2.0% w/w minocycline or a pharmaceutically acceptable salt thereof, about 0.1% to about 3% by weight of magnesium chloride, about 0.1% to about 0.2% by weight of sodium sulfite, about 4% to about 10% by weight of propylene glycol, about 4% to about 10% by weight of glycerin, about 0.5% to about 1.5% by weight of hydroxyl propyl methyl cellulose, and about 70% to about 85% of purified water.

In a further embodiment, there is provided a non-aqueous gel comprises from about 0.1% to about 0.3% w/w adapalene or a pharmaceutically acceptable salt thereof, from about 0.1 to about 10% w/w minocycline or a pharmaceutically acceptable salt thereof, about 40 to about 70% of gelling agent, and from about 10% to about 40% of non-aqueous solvent comprising light mineral oil, cetostearyl alcohol, and cyclomethicone.

In yet another embodiment, the aqueous gel composition comprises about 0.1% w/w adapalene or a pharmaceutically acceptable salt thereof and about 4.0% w/w minocycline or a pharmaceutically acceptable salt thereof, about 0.1% to about 3% by weight of magnesium chloride, about 0.1% to about 0.2% by weight of sodium sulfite, about 4% to about 10% by weight of propylene glycol, about 4% to about 10% by weight of glycerin, about 1% to about 2.5% by weight of sodium hyaluronate, and about 70% to about 85% of purified water.

In one embodiment, the aqueous gel composition comprises about 0.1% w/w adapalene or a pharmaceutically acceptable salt thereof and about 2% w/w minocycline or a pharmaceutically acceptable salt thereof, about 0.1% to about 3% by weight of magnesium chloride, about 0.1% to about 0.2% by weight of sodium sulfite, about 4% to about 10% by weight of propylene glycol, about 4% to about 10% by weight of glycerin, about 2% to about 4% by weight of acrylamide/sodium acryloyldimethyltaurate copolymer/isohexadecane/polysorbate, and about 70% to about 85% of purified water.

In another embodiment, the aqueous gel composition comprises about 0.1% w/w adapalene or a pharmaceutically acceptable salt thereof and about 4.0% w/w minocycline or a pharmaceutically acceptable salt thereof, about 0.1% to about 3% by weight of magnesium chloride, about 0.1% to about 0.2% by weight of sodium sulfite, about 4% to about 10% by weight of propylene glycol, about 4% to about 10% by weight of glycerin, about 2% to about 4% by weight of acrylamide/sodium acryloyldimethyltaurate copolymer/isohexadecane/polysorbate, and about 70% to about 85% of purified water.

In one or more embodiment, the topical composition is free, or substantially free, of a porous microcarrier having a hydrophobic surface, such as hydrophobic surface-modified silicon dioxide, porous polystyrene, porous polyamide, porous hydrophobic cellulose, porous polytetrafluoroethylene, or any combination of any of the foregoing.

In another embodiment, there is provided a method for making a composition by (a) combining minocycline, a magnesium compound, and an antioxidant in an aqueous solvent to form a mixture and agitating the mixture, (b) then dispersing or combining adapalene (e.g., in a solution) with the adapalene mixture, (c) followed by addition of a gelling agent and (d) further homogenizing the mixture to form a uniform mixture.

The topical composition may be prepared by combining or admixing the adapalene or a pharmaceutically acceptable salt thereof, minocycline or a pharmaceutically acceptable salt thereof, and other components. For example, a topical composition may be prepared by combining or admixing a gel comprising about 0.01 to about 1.0% w/w adapalene or a pharmaceutically acceptable salt thereof with a solution or dispersion comprising about 0.01 to about 20% w/w minocycline or a pharmaceutically acceptable salt thereof.

The topical compositions may be packaged in plastic tubes, metallic tubes, jars, automatic dispensing tubes, bottles, squeeze bottles, airless pump bottles and any other packaging material.

The topical composition of present invention may be provided in the form of a kit. The kits of the invention may contain a package and instructions for use. Instructions may be provided as separate from any container, but still contained in the kit. Alternatively, instructions may be located on a container, for example, on an exterior surface or on an interior surface.

The composition of the invention may be packaged separately in a single kit allowing simultaneous administration of the two compositions, or both components may be packaged in different kits.

In one embodiment adapalene and minocycline topical compositions are packaged separately and mixed together upon dispensing.

Components of the kit may be in the form of separate, sealed containers, such as bottles, jars, vials, ampules, tubes, and pouches, with the two containers usually being packaged together in the form of a kit. The kit may include instructions on how to admix the final product.

The pharmaceutical compositions of the present invention are useful for treating acne, such as moderate-to-severe cases of acne and acne vulgaris. One embodiment is a method of treating acne, such as acne vulgaris, in a patient comprising topically administering a pharmaceutical composition of the present invention to the patient. In one embodiment, a therapeutically effective amount of the pharmaceutical composition is administered. In one embodiment, the pharmaceutical composition is applied topically daily, e.g., one or more times daily, such as once daily, twice daily or three times daily.

Another embodiment is a method of treating a keratinization disorder in a patient comprising topically administering a pharmaceutical composition of the present invention to the patient. Keratinization disorders include, but are not limited to, acne conglobata, acne keloid on the back of the neck, acne medicamentosa, recurrent acne miliaria, acne necrotica, acne neonatorum, premenstrual acne, occupational acne, acne rosacea, senile acne, solar acne, acne vulgaris, comedonal acne, polymorphous acne, nodulocystic acne or secondary acne, such as solar acne. In one embodiment, inflammatory lesions (such as those due to acne vulgaris) are treated with the topical composition of the present invention.

One embodiment is a method of treating acne, comprising topically administering a gel composition comprising a fixed dose combination comprising about 0.1% to about 0.3% w/w adapalene or a pharmaceutically acceptable salt thereof, about 0.1 to about 10% w/w minocycline or a pharmaceutically acceptable salt thereof, about 10 to about 99% by weight of non-aqueous solvent or mixtures and about 0.05 to about 80% by weight of a gelling agent.

Another embodiment is a method of treating a keratinization disorder in a patient comprising topically administering a pharmaceutical composition of the present invention to the patient. Keratinization disorders include, but are not limited to, acne conglobata, acne keloid on the back of the neck, acne medicamentosa, recurrent acne miliaria, acne necrotica, acne neonatorum, premenstrual acne, occupational acne, acne rosacea, senile acne, solar acne, acne vulgaris, comedonal acne, polymorphous acne, nodulocystic acne or secondary acne, such as solar acne.

In Another embodiment present invention is of method of making a pharmaceutical composition for topical application which involves;
a. heating non-aqueous solvent or mixtures at a temperature of about 70-75° C.;
b. adding gelling agent to the heated mixture and homogenizing the mixture for about 10 min;
c. adding adapalene to the above mixture and homogenizing, to form a uniform mixture;
d. adding minocycline dispersed in mineral oil to the adapalene mixture and mixing both components to form a uniform dispersion.

### EXAMPLES

To further illustrate the invention, the following examples are provided. It is to be understood that these examples are provided for illustrative purpose and are not to be construed as limiting the scope of the invention.

### Example 1

### Adapalene Minocycline Hydrochloride Formulation

TABLE 1

| Ingredients | % w/w |
|---|---|
| Adapalene | 0.1-0.3 |
| Minocycline hydrochloride | 1-4 |
| Magnesium chloride | 0.1-3 |
| Sodium sulfite | 0.1-0.2 |
| Acrylamide/Sodium | 2-4 |
| acryloyldimethyltaurate | |
| copolymer/isohexadecane/polysorbate 80 | |
| Purified water | 90-95 |

Manufacturing Process:
1. Magnesium chloride is dissolved in water under stirring at 500-1000 RPM for about 10 min, till a clear solution is observed.
2. Sodium sulfite is added to the above solution under stirring at 500-1000 RPM for about 20 min, to obtain a clear solution.
3. Minocycline hydrochloride is added to the solution formed in to step 2 under stirring at 500-1000 RPM for 20-30 min.
4. Adapalene is added to the above mixture under stirring at 500-1000 RPM for 20-30 min. and the resultant mixture is homogenized to get a uniform dispersion.
5. Acrylamide/sodium acryloyldimethyltaurate copolymer/isohexadecane/polysorbate 80 is added to the mixture formed in step (4) and homogenized for about 30 min. to obtain a uniform gel dispersion.

### Example 2

### Adapalene/Minocycline Hydrochloride formulation

TABLE 2

| Ingredients | % w/w |
|---|---|
| Adapalene | 0.1-0.3 |
| Minocycline hydrochloride | 1-4 |
| Magnesium chloride | 0.1-3 |
| Sodium sulfite | 0.1-0.2 |
| Propylene Glycol | 4-10 |
| Glycerine | 4-10 |
| Acrylamide/Sodium | 2-4 |
| acryloyldimethyltaurate | |
| copolymer/isohexadecane/polysorbate 80 | |
| Purified water | 70-85 |

Manufacturing Process:
1. Magnesium chloride is dissolved in purified water under stirring at 500-1000 RPM for about 10 min, till a clear solution is observed.
2. Sodium sulfite is added to the above solution under stirring at 500-1000 RPM for about 20 min, to obtain a clear solution.
3. Minocycline hydrochloride is added to the solution formed in to step (2) under stirring at 500-1000 RPM for 20-30 min to ensure complete dissolution of drug in solvent.
4. Adapalene is dispersed in a solvent system comprising glycerin and propylene glycol under stirring at 500-1000 RPM for 20 min to get uniform dispersion of API.
5. Adapalene dispersion is then added to the solution mixture formed in step (3) under stirring at 500-1000 RPM for 20-30 mins.
6. Acrylamide/sodium acryloyldimethyltaurate copolymer/isohexadecane/polysorbate 80 is added to the mixture formed in step (5) and homogenized for about 30 min. to obtain a uniform gel dispersion.

### Example 3

### Adapalene/Minocycline Hydrochloride Formulation

TABLE 3

| Ingredients | % w/w |
|---|---|
| Adapalene | 0.1-0.3 |
| Minocycline hydrochloride | 1-4 |
| Magnesium chloride | 0.1-3 |
| Sodium sulfite | 0.1-0.2 |
| Propylene glycol | 4-10 |
| Glycerine | 4-10 |
| Sodium hyaluronate | 1-2.5 |
| Purified water | 70-85 |

Manufacturing Process:
1. Magnesium chloride is dissolved in purified water under stirring at 500-1000 RPM for about 10 min, till a clear solution is observed.
2. Sodium Sulfite is added to the above solution under stirring at 500-1000 RPM for about 20 min, to obtain a clear solution.
3. Minocycline hydrochloride is added to the solution formed in to step (2) under stirring at 500-1000 RPM for about 20-30 min to ensure complete dissolution of drug in solvent.
4. Sodium hyaluronate is added and dispersed in to solution mixture formed in step (3) under stirring at 1000-2000 RPM for about 30-60 mins to get a yellow colored clear gel.
5. Adapalene is dispersed in a solvent system comprising glycerin and propylene glycol under stirring at 500-1000 RPM for about 20 min to get uniform dispersion of API.
6. Adapalene dispersion is then added to the solution mixture formed in step (4) and homogenized for about 20-30 mins to obtain a uniform gel dispersion.

### Example 4

### Adapalene/Minocycline Hydrochloride Formulation

TABLE 4

| Ingredients | % w/w |
|---|---|
| Adapalene | 0.1-0.3 |
| Minocycline hydrochloride | 1-4 |
| Magnesium chloride | 0.1-3 |
| Sodium sulfite | 0.1-0.2 |
| Propylene glycol | 4-10 |
| Glycerine | 4-10 |
| Hydroxy propyl methyl cellulose | 0.5-1.5 |
| Purified water | 70-85 |

Manufacturing Process:
1. Magnesium chloride is dissolved in purified water under stirring at 500-1000 RPM for about 10 min, till a clear solution is observed.
2. Sodium sulfite is added to the above solution under stirring at 500-1000 RPM for about 20 min, to obtain a clear solution.
3. Minocycline hydrochloride is added to the solution formed in to step (2) under stirring at 500-1000 RPM for 20-30 min to ensure complete dissolution of drug in solvent.
4. Hydroxy propyl methyl cellulose is added to the solution mixture formed in step (3 under stirring at 1000-2000 RPM for about 30-60 mins to get a yellow colored clear gel.
5. Adapalene is dispersed in a solvent system comprising glycerin and propylene glycol under stirring at 500-1000 RPM for 20 min to get uniform dispersion of API.
6. Adapalene dispersion is then added to the solution mixture formed in step (4) and homogenized for about 20-30 mins to obtain a uniform gel dispersion.

### Example 5

### Adapalene/Minocycline Hydrochloride Formulation

TABLE 5

| Ingredients | % w/w |
|---|---|
| Adapalene | 0.1-0.3 |
| Minocycline hydrochloride | 1-4 |
| Magnesium chloride | 0.1-3 |
| Sodium sulfite | 0.1-0.2 |
| Propylene glycol | 4-10 |
| Glycerine | 4-10 |
| Hydroxy ethyl cellulose | 1-1.75 |
| Purified water | 70-85 |

Manufacturing Process:
1. Magnesium chloride is dissolved in purified water under stirring at 500-1000 RPM for about 10 min, till a clear solution is observed.
2. Sodium sulfite is added to the above solution under stirring at 500-1000 RPM for about 20 min, to obtain a clear solution.
3. Minocycline hydrochloride is added to the solution formed in to step (2) under stirring at 500-1000 RPM for about 20-30 min to ensure complete dissolution of drug in solvent.
4. Hydroxy ethyl cellulose is added to the solution mixture formed in step (3) under stirring at 1000-2000 RPM for about 30-60 mins to get a yellow colored clear gel.
5. Adapalene is dispersed in a solvent system comprising glycerin and propylene glycol under stirring at 500-1000 RPM for about 20 min to get uniform dispersion of API.
6. Adapalene dispersion is then added to the solution mixture formed in step (4) and homogenized for about 20-30 mins to obtain a uniform gel dispersion.

### Example 6

### Adapalene/Minocycline Hydrochloride Formulation

TABLE 6

| Ingredients | % w/w |
|---|---|
| Adapalene | 0.1-0.3 |
| Minocycline hydrochloride | 1-4 |
| Magnesium chloride hexahydrate | 0.1-10 |
| Sodium sulfite | 0.1-1.0 |
| Propylene glycol | 2-20 |
| Glycerine | 2-15 |
| Sodium hyaluronate | 1-10 |
| Purified water | 70-85 |

Manufacturing Process:

Preparation of Adapalene Aqueous Gel:
1. Magnesium chloride is dissolved in purified water under stirring at 500-1000 RPM for about 10-20 min, till a clear solution is observed.
2. Sodium Sulfite is added to the above solution under stirring at 500-1000 RPM for about 10-30 min, to obtain a clear solution.
3. Sodium hyaluronate is added and dispersed in to solution mixture formed in step (2) under stirring at 1000-2000 RPM for about 30-60 mins to get a yellow colored clear gel.
4. Adapalene is dispersed in a solvent system comprising glycerin and propylene glycol under stirring at 500-1000 RPM for about 20-30 min to get uniform dispersion of API.
5. Adapalene dispersion is then added to the gel formed in step (3) and homogenized for about 20-30 mins to obtain a uniform gel dispersion.

Minocycline Hydrochloride Powder Filling in Vial:
1. Minocycline Hydrochloride powder is filled in 5 ml vials.

Packaging of Both Formulations:
1. Adapalene aqueous gel and minocycline hydrochloride powder are packaged separately and mixed together just prior to or during administration.

Dispensing:
1. Indicated amount of purified water is added to the vial to completely dissolve minocycline hydrochloride. If needed additional amount of purified water is added to further dissolve minocycline hydrochloride.
2. Minocycline hydrochloride solution prepared in step (1) is added to the adapalene gel and mixed for about 1-2 mins to produce a final mixed product.

### Example 7

### Adapalene/Minocycline Hydrochloride Formulation

TABLE 7

| Ingredients | % w/w |
|---|---|
| Adapalene | 0.1-0.3 |
| Minocycline Hydrochloride | 1-4 |
| Cyclomethicone | 5-13 |
| Light mineral Oil | 4-15 |
| Bees wax or Silky wax | 1-3 |
| Cetostearyl alcohol | 1-8 |
| cyclopentasiloxane and dimethicone cross polymer | 55-85 |

Manufacturing Process
1. Cyclomethicone, bees wax/silky wax 10, cetostearyl alcohol were added to a stainless steel vessel and heated to 70-75° C.
2. Cyclopentasiloxane and dimethicone cross polymer was added to the above mixture and homogenized for about 10 min.
3. Adapalene was added to the above mixture and the resulting mixture was homogenized at temperature (70-75° C.) for about 15 min, to form a homogenized phase (I).
4. In a separate vessel, mineral oil was heated to 40-45° C., and minocycline hydrochloride was dispersed in mineral oil under continuous stirring, to obtain a homogenized phase (II).
5. The homogenized phase (II) formed in step (4) was added to the phase (I) formed in step (3), and both phases were mixed together for about 20 minutes to obtain a uniform gel dispersion.

### Example 8

### Adapalene/Minocycline Hydrochloride Formulation

TABLE 8

| Ingredients | % w/w |
|---|---|
| Adapalene | 0.1-0.3 |
| Minocycline hydrochloride | 1-4 |
| Cyclomethicone | 5-13 |
| Light mineral oil | 4-15 |
| Cetostearyl alcohol | 1-8 |
| cyclopentasiloxane and dimethicone cross polymer | 55-85 |

Manufacturing Process
1. Cyclomethicone, cetostearyl alcohol were added to a stainless steel vessel and heated to 70-75° C.
2. Cyclopentasiloxane and dimethicone cross polymer was added to the above mixture and the temperature of vessel was maintained at 70-75° C. The mixture was homogenized for about 10 min to obtain a translucent gel.
3. Adapalene was added to the above mixture and the resulting mixture was homogenized at a temperature 50-55° C. for about 15 minutes, to form a homogenized phase (I).
4. In a separate vessel, mineral oil was heated to 40-45° C., and minocycline hydrochloride was dispersed in the light mineral oil under continuous stirring, for about 20-30 minutes to obtain a homogenized phase (II).
5. The homogenized phase (II) formed in step (4) was added to the phase (I) formed in step (3), and both phases were mixed together for about 20 minutes. Stirring was continued to obtain a uniform gel dispersion.

### Example 9

### Adapalene/Minocycline Hydrochloride Formulation

TABLE 9

| Ingredients | % w/w |
|---|---|
| Adapalene | 0.1-0.3 |
| Minocycline Hydrochloride | 1-4 |
| Cyclomethicone | 5-13 |
| Light mineral Oil | 4-15 |
| Bees wax or Silky wax | 1-3 |
| Isopropyl Myristate | 1-5 |
| Cetostearyl alcohol | 1-8 |
| Cyclopentasiloxane and dimethicone cross polymer | 55-85 |

Manufacturing Process
1. Cyclomethicone, bees wax/silky wax 10, cetostearyl alcohol were added to a stainless steel vessel and heated to 70-75° C.
2. Cyclopentasiloxane and dimethicone cross polymer was added to the above mixture and the temperature of vessel was maintained at 70-75° C. The mixture was homogenized for about 10 minutes to obtain a translucent gel.
3. Adapalene was added to the above mixture and the resulting mixture was homogenized at temperature 50-55° C. for about 15 minutes, to form a homogenized phase (I).
4. In a separate vessel, mineral oil was heated up to 40-45° C., and minocycline hydrochloride was dispersed in the mineral oil under continuous stirring for about 20-30 minutes to obtain an homogenized phase (II). 5. The homogenized phase (II) formed in step (4) was added to the phase (I) formed in step (3), and both phases were mixed together and the mixture was homogenized for about 20 minutes to obtain a uniform gel dispersion.
6. Isopropyl myristate was heated up to 40-45° C. in a separate vessel, and then added to the uniform gel dispersion formed in step 5, under homogenization at 40-45° C. Stirring was continued for 5-10 about minutes to obtain a final uniform gel dispersion.

### Example 10

### Adapalene/Minocycline Hydrochloride Formulation

TABLE 10

| Ingredients | % w/w |
|---|---|
| Adapalene | 0.1-0.3 |
| Minocycline hydrochloride | 1-4 |
| Cyclomethicone | 5-13 |
| Light mineral Oil | 4-15 |
| Isopropyl myristate | 1-5 |
| Cetostearyl alcohol | 1-8 |
| Cyclopentasiloxane and dimethicone cross polymer | 55-85 |

Manufacturing Process
1. Cyclomethicone, and cetostearyl alcohol were added to a stainless steel vessel and heated to 70-75° C.
2. Cyclopentasiloxane and dimethicone cross polymer was added to the above mixture and the temperature of vessel was maintained at 70-75° C. The mixture was homogenized for about 10 minutes to obtain a translucent gel.
3. Adapalene was added to the above mixture and the resulting mixture was homogenized at temperature 50-55° C. for about 15 minutes, to form an homogenized phase (I).
4. In a separate vessel, mineral oil was heated up to 40-45° C., and minocycline hydrochloride was dispersed in the mineral oil under continuous stirring for about 20-30 minutes to obtain an homogenized phase (II). The temperature was maintained at 40-45° C.
5. The homogenized phase (II) formed in step (4) was added to the phase (I) formed in step (3). Both phases were mixed together at 5000-6000 rpm and the temperature of the vessel was maintained at about 40-45° C. The mixture was homogenized for about 20 minutes. Stirring was continued until the temperature reached 28-32° C. to obtain a uniform gel dispersion.
6. Isopropyl myristate was heated up to 40-45° C. in a separate vessel, and then added to the uniform gel dispersion formed in step (5), under homogenization at 40-45° C. Stirring was continued for 5-10 about minutes till the temperature reached 28-32° C. to obtain a final uniform gel dispersion.

### Example 11

### Adapalene/Minocycline Hydrochloride Formulation

TABLE 11

| Ingredients | % w/w |
|---|---|
| Adapalene | 0.1-0.3 |
| Minocycline hydrochloride | 1-4 |
| Cyclomethicone | 5-13 |
| Light mineral Oil | 4-15 |
| Bees wax | 1-3 |
| Isopropyl myristate | 1-5 |
| Cyclopentasiloxane and dimethicone cross polymer | 55-85 |

Manufacturing Process
1. Cyclomethicone, and bees wax were added to a stainless steel vessel and the mixture heated to 70-75° C.
2. Cyclopentasiloxane and dimethicone cross polymer was added to the above mixture and the temperature of vessel was maintained at 70-75° C. The mixture was homogenized for about 10 minutes to obtain a translucent gel.
3. Adapalene was added to the above mixture and the resulting mixture was homogenized at temperature 50-55° C. for about 15 minutes, to form a homogenized phase (I).
4. In a separate vessel, mineral oil was heated up to 40-45° C., and minocycline hydrochloride was dispersed in the mineral oil under continuous stirring for about 20-30 minutes to obtain an homogenized phase (II). The temperature was maintained at 40-45° C.
5. The homogenized phase (II) formed in step (4) was added to the phase (I) formed in step (3). Both phases were mixed together at 5000-6000 rpm and the temperature of the vessel was maintained at about 40-45° C. The mixture was homogenized for about 20 minutes. Stirring was continued until the temperature reached 28-32° C. to obtain a uniform gel dispersion.
6. Isopropyl myristate was heated up to 40-45° C. in a separate vessel, and then added to the uniform gel dispersion formed in step (5), under homogenization at 40-45° C. Stirring was continued for 5-10 about minutes until temperature reached 28-32° C. to afford a final uniform gel dispersion.

### Example 12

### Adapalene/Minocycline Hydrochloride Formulation

TABLE 12

| Ingredients | % w/w |
|---|---|
| Adapalene | 0.1-0.3 |
| Minocycline hydrochloride | 1-4 |
| Cyclomethicone | 5-13 |
| Light mineral Oil | 4-15 |
| Cyclopentasiloxane and dimethicone cross polymer | 55-85 |

Manufacturing Process
1. Cyclomethicone was added to a stainless steel vessel and heated to 70-75° C.
2. Cyclopentasiloxane and dimethicone cross polymer was added to the above mixture and the temperature of vessel was maintained at 70-75° C. The mixture was homogenized for about 10 minutes to obtain a translucent gel.
3. Adapalene was added to the above mixture and the resulting mixture was homogenized at 50-55° C. for about 15 minutes to form a homogenized phase (I).
4. In a separate vessel, mineral oil was heated up to 40-45° C., and minocycline hydrochloride was dispersed in the mineral oil under continuous stirring for about 20-30 minutes to obtain a homogenized phase (II). The temperature was maintained at 40-45° C.
5. The homogenized phase (II) formed in step (4) was added to the phase (I) formed in step (3). Both phases were mixed together at 5000-6000 rpm and the temperature of the vessel was maintained at about 40-45° C. The mixture was homogenized for about 20 minutes. Stirring was continued until the temperature reached 28-32° C. to obtain a uniform gel dispersion.

### Example 13

### Adapalene/Minocycline Hydrochloride Formulation

TABLE 13

| Ingredients | % w/w |
|---|---|
| Adapalene | 0.1-0.3 |
| Minocycline hydrochloride | 1-4 |
| Cyclomethicone | 5-13 |
| Light mineral Oil | 4-15 |
| Cetostearyl Alcohol | 1-8 |
| Cyclopentasiloxane and dimethicone cross polymer | 55-85 |

Manufacturing Process
1. Cyclomethicone, and cetostearyl alcohol were added to a stainless steel vessel and heated up to 70-75° C.
2. Cyclopentasiloxane and dimethicone cross polymer was added to the above mixture and the temperature of vessel was maintained at 70-75° C. The mixture was homogenized for about 10 minutes to obtain a translucent gel.
3. Adapalene was added to the above mixture and the resulting mixture was homogenized at 50-55° C. for about 15 minutes, to form a homogenized phase (I).
4. In a separate vessel, mineral oil was heated up to 40-45° C., and minocycline hydrochloride was dispersed in the mineral oil under continuous stirring for about 20-30 minutes to obtain a homogenized phase (II). The temperature was maintained at 40-45° C.
5. The homogenized phase (II) formed in step (4) was added to the phase (I) formed in step (3). Both phases were mixed together at 5000-6000 rpm and the temperature of the vessel was maintained at about 40-45° C. The mixture was homogenized for about 20 minutes Stirring was continued until the temperature reached 28-32° C. to obtain a uniform gel dispersion.

1. A pharmaceutical composition comprising adapalene or a pharmaceutical acceptable salt thereof and minocycline or a pharmaceutical acceptable salt thereof, wherein the composition is for topical application.

2. The pharmaceutical composition of claim 1, wherein the compositions is a fixed dose combination of adapalene or a pharmaceutical acceptable salt thereof and minocycline or a pharmaceutical acceptable salt thereof.

3. The pharmaceutical composition of claim 1, wherein the composition is in the form of a cream, gel, lotion, ointment, liniment, paste, oil-in-water emulsion, water-in-oil emulsion and foam.

4. The pharmaceutical composition of claim 1, wherein the composition is in the form of a topical gel.

5. The pharmaceutical composition of claim 2, wherein the composition is an aqueous pharmaceutical composition.

6. The pharmaceutical composition of claim 2, wherein the composition is a non-aqueous pharmaceutical composition.

7. The pharmaceutical composition of claim 2, wherein the adapalene or pharmaceutical acceptable salt thereof is present in an amount of from about 0.1% to about 1% w/w and minocycline or pharmaceutical acceptable salt thereof is present in an amount of from about 0.01 to about 20% w/w.

8. The pharmaceutical composition of claim 5, wherein the composition further comprises one or more pharmaceutical excipients selected from a magnesium compound, solvent, gelling agent, preservative, buffer, penetration enhancer, pH adjusting agent, emollient, humectant, antioxidant and chelating agents.

9. The pharmaceutical composition of claim 6, wherein the composition further comprises one or more pharmaceutical excipients selected from a solvent, gelling agent, preservative, buffer, penetration enhancer, pH adjusting agent, emollient, humectant, antioxidant and chelating agents.

10. The pharmaceutical composition of claim 2, wherein 4-epi minocycline is not more than 3%, by weight when stored at 40° C. and 75% RH for one month, based upon the total weight of minocycline and pharmaceutically acceptable salts thereof prior to storage.

11. The pharmaceutical composition of claim 8, wherein the gelling agent comprises one or more of a cellulose polymer, a cellulose derivative, crosslinked acrylic acid polymers, acrylamide/sodium acryloyldimethyltaurate copolymer/isohexadecane/polysorbate 80, cyclopentasiloxane and dimethicone cross polymer, polyvinyl alcohol, poloxamer, polysaccharides, polyvinyl pyrrolidone and gums.

12. The pharmaceutical composition of claim 8, wherein the magnesium compound comprises one or more of magnesium chloride, magnesium acetate, magnesium sulfate, magnesium carbonate, magnesium gluconate, and magnesium chloride hexahydrate.

13-17. (canceled)

18. The pharmaceutical composition of claim 9, wherein the gelling agent comprises one or more cellulose polymer, a cellulose derivative, crosslinked acrylic acid polymers, cyclopentasiloxane and dimethicone cross polymer, polyvinyl alcohol, poloxamer, polysaccharides, polyvinyl pyrrolidone and gums.

19. The pharmaceutical composition of claim 2, wherein the adapalene or pharmaceutical acceptable salt thereof is present in an amount of from about 0.1% w/w to about 1% w/w and minocycline or pharmaceutical acceptable salt thereof is present in an amount of from about 1% w/w to about 10% w/w.

20. The pharmaceutical composition of claim 2, wherein the adapalene or pharmaceutical acceptable salt thereof is present in an amount of about 0.1% w/w to about 0.3% w/w and minocycline or pharmaceutical acceptable salt thereof is present in an amount of from about 2% w/w to 4% w/w.

21. A pharmaceutical composition for topical application comprising:
a. from about 0.1% w/w to about 0.3% w/w adapalene or a pharmaceutically acceptable salt thereof;
b. about 1.0% w/w to about 10% w/w minocycline or a pharmaceutically acceptable salt thereof;
c. a gelling agent;
d. a silicone oil;
e. a non-aqueous solvent; and
f. optionally, one or more pharmaceutically acceptable excipient thereof;
wherein the composition is a non-aqueous pharmaceutical composition.

22. The pharmaceutical composition of claim 21, wherein the composition comprises fixed dose combination of adapalene or a pharmaceutically acceptable salt thereof and minocycline or a pharmaceutically acceptable salt thereof.

23. A pharmaceutical composition for topical application comprising:
a. from about 0.1% w/w to about 0.3% w/w adapalene or a pharmaceutically acceptable salt thereof;
b. from about 1.0% w/w to about 10% w/w minocycline or a pharmaceutically acceptable salt thereof;
c. from about 40% w/w to about 70% w/w of a gelling agent selected from cyclopentasiloxane and dimethicone cross polymer;
d. a silicone oil selected from dimethicone, cyclomethicone, polyalkyl siloxane, polyaryl siloxane, polyalkylaryl siloxane, a polyether siloxane copolymer and a poly(dimethylsiloxane)-(diphenyl-siloxane) copolymer;
e. from about 10% w/w to about 99% w/w a non-aqueous solvent selected from mineral oil, light mineral oil, triglycerides, medium chain triglyceride, capric/caprylic triglyceride isopropyl palmitate, isopropyl myristate, isopropyl isostearate, poly propylene glycol 15-stearly ether, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, and maleated soybean oil; and
f. optionally, one or more pharmaceutically acceptable excipient thereof.

24. A method for treating acne in a subject in need thereof comprising topically administering a pharmaceutical composition comprising:
a. about 0.01% w/w to about 1.0% w/w adapalene or a pharmaceutically acceptable salt thereof;
b. about 0.01% w/w to about 20% w/w minocycline or a pharmaceutically acceptable salt thereof;
c. about 10% w/w to about 99% w/w by weight of non-aqueous solvent or mixtures; and
d. about 0.05% w/w to about 75% w/w of gelling agent.

25. The method of claim 24, wherein the acne is selected from acne conglobata, acne keloid on the back of the neck, acne medicamentosa, recurrent acne miliaria, acne necrotica, acne neonatorum, premenstrual acne, occupational acne, acne rosacea, senile acne, solar acne, acne vulgaris, comedonal acne, polymorphous acne, nodulocystic acne, secondary acne, and any combination of any of the foregoing.

26. The method of claim 25, wherein the acne is moderate-to-severe acne vulgaris.

27. The method of claim 25, wherein the acne is inflammatory lesions of acne vulgaris.

28. A pharmaceutical composition for topical application comprising:
a. from about 1.0% w/w to about 10% w/w minocycline or a pharmaceutically acceptable salt thereof;
b. from about 40% w/w to about 70% w/w of a gelling agent selected from cyclopentasiloxane and dimethicone cross polymer;
c. a silicone oil selected from dimethicone, cyclomethicone, polyalkyl siloxane, polyaryl siloxane, polyalkylaryl siloxane, a polyether siloxane copolymer and a poly(dimethylsiloxane)-(diphenyl-siloxane) copolymer; and
d. from about 10% w/w to about 99% w/w a non-aqueous solvent selected from mineral oil, light mineral oil, triglycerides, medium chain triglyceride, capric/caprylic triglyceride isopropyl palmitate, isopropyl myristate, isopropyl isostearate, poly propylene glycol 15-stearly ether, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, and maleated soybean oil; and
e. optionally, one or more pharmaceutically acceptable excipient thereof.

29. The composition of claim 28, wherein the composition comprises from about 2% w/w to about 4% w/w minocycline or a pharmaceutically acceptable salt thereof.
